(19) United States
(12) Patent Application Publication
(10) Pub. No.: US 2022/0127231 A1
(43) Pub. Date: Apr. 28, 2022

(54) PROCESS FOR THE PREPARTION OF AMIFAMPRIDINE PHOSPHATE

(71) Applicants: RAFIUDDIN Dr, Raichur (IN); YUVATEJA BOYINA, Raichur (IN)

(72) Inventors: RAFIUDDIN Dr, Raichur (IN); YUVATEJA BOYINA, Raichur (IN)

(73) Assignee: SHILPA MEDICARE LTD, RAICHUR (IN)

(21) Appl. No.: 17/474,087

(22) Filed: Sep. 14, 2021

(30) Foreign Application Priority Data
Oct. 27, 2020 (IN) 202041046824

(51) Int. Cl. C07D 213/73

(52) U.S. Cl. CPC C07D 213/73

(57) ABSTRACT

The present invention relates to an improved process for the preparation of substantially pure Amifampridine phosphate

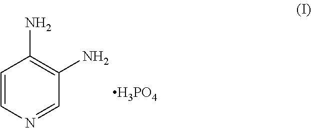

The present invention further relates to a substantially pure Amifampridine Phosphate, useful for the preparation of pharmaceutical composition thereof.

### FIELD OF THE INVENTION

The present invention relates to an improved process for the preparation of substantially pure Amifampridine phosphate.

The present invention further relates to a substantially pure Amifampridine Phosphate, useful for the preparation of pharmaceutical composition thereof.

### BACKGROUND OF THE INVENTION

Amifampridine is chemically known as 3,4-diaminopyridine. The structural formula of Amifampridine is as described in Formula (II).

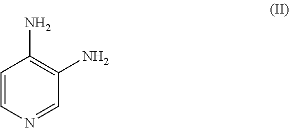

Amifampridine has an empirical formula C₅H₇N₃ and a molecular weight of 109.13. Amifampridine is marketed in the form of Amifampridine phosphate. Amifampridine phosphate is a white crystalline powder with the empirical formula C₅H₇N₃ and a molecular weight of 109.13. It is practically soluble in water, slightly soluble in dimethyl sulfoxide, glacial acetic acid and methanol and very slightly soluble in ethanol and dimethylformamide. No stereoisomerism is observed. The pure drug substance melts at 229±2° C.; pH of 1% solution in water at 25° C. is 4.6±0.2. Amifampridine is marketed under the brand name Zenas® as an oral tablet.

Amifampridine well knows to block potassium channels in the nerve cells. This blockage causes depolarisation of the presynaptic membrane and slows down or inhibits repolarisation. Prolonged depolarisation induces the opening of slow voltage-gated calcium channels (VGCC) and the influx of calcium. The increased concentration of intracellular calcium induces exocytosis of the synaptic vesicles containing acetylcholine (Ach). It has been proposed that treatment with Amifampridine counter acts the pathologically-reduced ACh release in patients with Lambert-Eaton syndrome (LEMS) and hence provides symptomatic relief to the patient. These pharmacological actions support the proposed clinical indication.

Amifampridine is also known to be used in treatments related to many of the congenital myasthenic syndromes, particularly those with defects in choline acetyl transferase, downstream kinase and those where any kind of defect causes “fast channel” behaviour of the acetylcholine receptor.

EP0156495A1 disclosed a process for the preparation of Amifampridine starting from 3-nitro-4-benzylaminopyridine. The process is as demonstrated below:

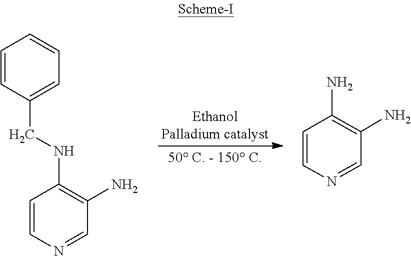 [SMILES: Nc1cnccc1NCc1ccccc1 ; Nc1ccncc1N]

This process involves deprotection of benzyl protected amino group at higher temperature, which is not feasible at large scale production and with lower yields resulting in the formation of byproducts/impurities

EP0159112A1 disclosed different process for the preparation of Amifampridine mono hydrochloride starting from 3-nitro-4-chloropyridine. The processes disclosed are as demonstrated below in Scheme-II and III:

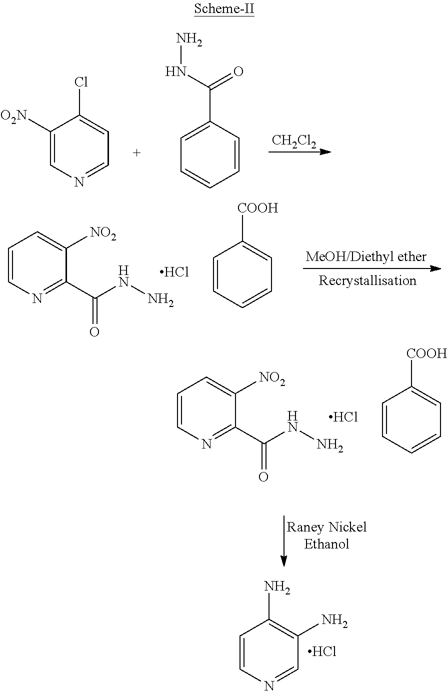

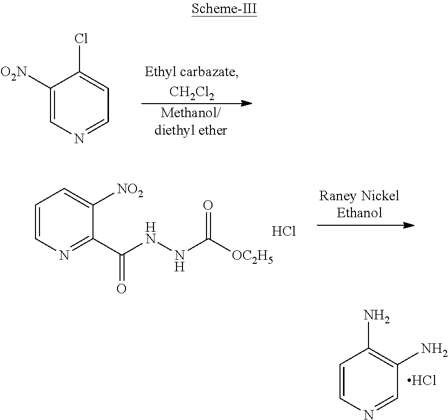

Guyon, Francois et al in US20040106651A1 discloses tartrate and phosphate salts of Amifampridine as only stable salts. It was observed that the precipitation of amifampridine phosphate immediately after addition of phosphoric acid. However, the isolation of phosphate salt of amifampridine is difficult due to its high solubility in water, which reduces the yield.

Journal of Heterocyclic Chemistry (1999), 36(5), 1143-1145 discloses a process in for the preparation of 3,4-Diaminopyridine starting from 3-nitropyridine. The process is as demonstrated below:

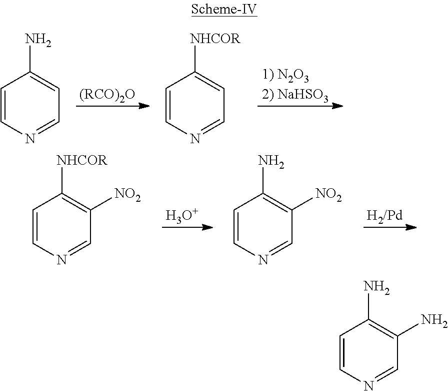

EP 3696169 A1 disclosed a process for the preparation of Amifampridine Phosphate, comprising dissolving amifampridine in an alcoholic solvent followed by addition of phosphoric acid solution. The process disclosed yields in amifampridine phosphate, having a triamine impurity (Impurity C) of greater than 0.1%.

After review of various literatures, and reproducing some of the processes, following were apparent disadvantages in different processes were noticed, which makes many such reported processes not amenable to scale up.
In most of the patent literatures, hazardous chemical reagents are used, which are not feasible and cumbersome to handle on industrial scale.
Unwanted side/parallel reactions are observed during the formation of 3,4-Diaminopyridine, due to the use of higher temperature reaction conditions. Further, the use of higher temperature is not industrially feasible.
Expensive Reducing agents like Pd/C is used during reduction of —NO₂ group to amino group in solvents is very difficult to handle at commercial level and makes the process commercially non-viable.

In view of the above and to overcome the prior-art disadvantages, the present inventors have now developed an improved process for the preparation of substantially pure Amifampridine, using minimal steps, which is industrially feasible, with the use of industrial friendly solvents, which does not involve tedious work up and hazardous steps.

### OBJECTIVE OF THE INVENTION

The main objective of the invention is to provide a stable & highly pure Amifampridine phosphate salt.

Yet another objective of the invention is to provide an improved process for the preparation of substantially pure Amifampridine phosphate.

Yet another objective of the invention is to provide an improved process for the preparation of substantially pure Amifampridine phosphate having purity of greater than 99.5%.

### SUMMARY OF THE INVENTION

The present invention relates to an improved process for the preparation of substantially pure Amifampridine phosphate of Formula (I)

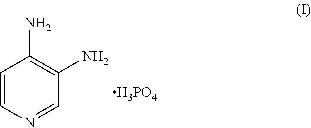

comprising the steps of:
a) reacting 4-amino pyridine (III) with a nitrating agent in presence/absence of a solvent to yield 4-Amino-3-Nitro Pyridine (IV);

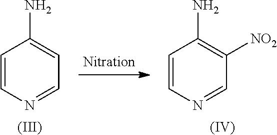

b) reacting 4-Amino-3-Nitro Pyridine (IV) with a reducing agent to provide Amifampridine (II); and

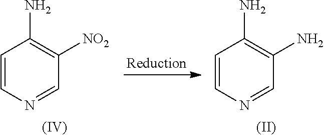

c) converting Amifampridine to Amifampridine phosphate salt comprises:
   i. preparing a non-alcoholic solution of amifampridine;
   ii. adding phosphoric acid solution to non-alcoholic solution obtained in step (i); and
   iii. optionally adding anti solvent to yield highly pure Amifampridine phosphate having a purity of greater than 99.5% (by HPLC).

In another aspect of the present invention relates substantially pure Amifampridine phosphate, wherein in the salt is obtained by the process comprising:
a) preparing a non-alcoholic solution of amifampridine;
b) adding phosphoric acid solution to non-alcoholic solution obtained in step (i); and
c) optionally adding anti solvent to yield highly pure Amifampridine phosphate having a purity of greater than 99.5% (by HPLC).

In another aspect of the present invention relates Substantially pure Amifampridine phosphate, having a purity of greater than 99.5% and total impurities A to D collectively less than 0.5% by HPLC and specifically impurity-C less than 0.1%.

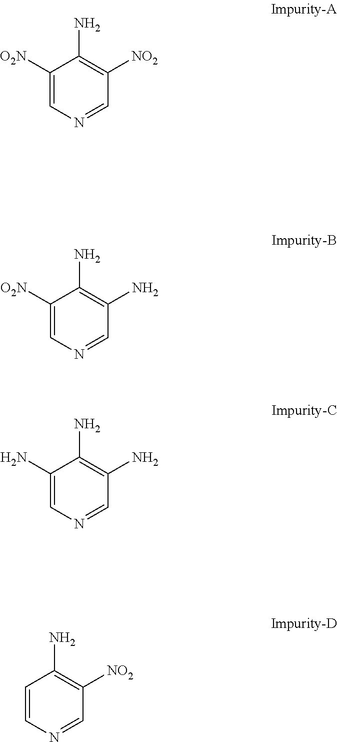 [SMILES: Nc1c([N+](=O)[O-])cncc1[N+](=O)[O-] ; Nc1cncc([N+](=O)[O-])c1N ; Nc1cncc(N)c1N ; Nc1ccncc1[N+](=O)[O-]]

### DETAILED DESCRIPTION OF THE INVENTION

The present invention relates to an improved process for the preparation of substantially pure Amifampridine phosphate of Formula (I) comprising the steps of nitrating 4-amino pyridine (III) using a nitrating agent in presence/absence of a solvent acids such as sulfuric acid, acetic acid; nitriles such acetonitrile; nitro methane; alcohols such as C1-5 alcohols; Glycol ethers such as 2-Butoxyethanol, Diglyme, Dimethoxyethane, 2-Ethoxy ethanol, 2-(2-Ethoxyethoxy)ethanol, 2-Methoxyethanol, 2-(2-Methoxyethoxy) ethanol, Octaethylene glycol monododecyl ether, Pentaethylene glycol monododecyl ether, Phenoxyethanol, Propylene glycol methyl ether acetate, Tetraethylene glycol dimethyl ether, Triethylene glycol, Triethylene glycol dimethyl ether; water or mixtures thereof; in presence of nitrating agent selected from nitric acid, nitric acid+sulfuric acid, sodium nitrate, potassium nitrate, silver nitrate, Methyl nitrate, nitric acid+sulfuric acid+urea; at a temperature ranging from 40 to 90° C. The reaction mixture was stirred for a period of 4 hours to 8 hours to yield 4-Amino-3-Nitro Pyridine (IV)

The present inventors found that the use of potassium nitrate as a nitrating agent completes the reaction at a temperature ranging from 40-45° C., which is industrially feasible, cost effective and avoids unwanted reactions, which minimized the formation of impurity. After completion of the reaction, the removal of potassium nitrate is modest and does not require any cumbersome workup.

The above obtained 4-Amino-3-Nitro Pyridine (IV) was reduced in presence of a reducing agent selected from hydrogen sulfide, sodium sulfide, potassium sulfide, sodium hydrogen sulfide, Zn/Magnesium in presence of Hydrazine hydrate or a mixture thereof; at a temperature ranging from 30-75° C. in presence of a solvent-2 selected from acids such as sulfuric acid, acetic acid; nitriles such acetonitrile; nitro methane; alcohols such as C1-5 alcohols; Glycol ethers such as 2-Butoxyethanol, Diglyme, Dimethoxyethane, 2-Ethoxy ethanol, 2-(2-Ethoxyethoxy)ethanol, 2-Methoxyethanol, 2-(2-Methoxyethoxy) ethanol, Octaethylene glycol monododecyl ether, Pentaethylene glycol monododecyl ether, Phenoxyethanol, Propylene glycol methyl ether acetate, Tetraethylene glycol dimethyl ether, Triethylene glycol, Triethylene glycol dimethyl ether; water or mixtures thereof. The obtained reaction mixture was stirred at a temperature ranging from 35-75° C. for 30 minutes −1 hour. To the obtained reaction mass water was cooled and stirred for 30 min to 3 hours. Filtered the solid and washed the wet cake with to provide Amifampridine

The present inventors surprisingly found that the use of sulfide reducing agents in the reduction step leads to the formation of pure Amifampridine, which is devoid of other process related impurities. Further, the prior-art process utilizes palladium carbon in reduction step. However, the Amifampridine obtained is not much pure and the reaction is incomplete leads to the formation of lower yields as well unwanted by-products.

The obtained Amifampridine was optionally purified by treating the residue with a suitable solvent selected from, but are not limited to: alcohols, such as C2-C6 alcohols like ethanol, 1-propanol, 2-propanol (isopropyl alcohol), 1-butanol, 2-butanol, t-butyl alcohol; or nitriles, such as acetonitrile or propionitrile; amides such as N,N-dimethylformamide, N,N-dimethylacetamide, N-methyl-2-pyrrolidone; sulfoxides such as dimethylsulfoxide; halogenated hydrocarbons such as dichloromethane; aromatic hydrocarbons such as toluene, xylene; esters such as ethyl acetate, n-propyl acetate, n-butyl acetate, isopropyl acetate, isobutyl acetate, t-butyl acetate; ethers such as diethyl ether, diisopropyl ether, methyl t-butyl ether, tetrahydrofuran, 1,4-dioxane, 2-methoxyethanol, anisole; ketones such as acetone, ethyl methyl ketone, diethyl ketone, methyl isobutyl ketone; organic solvents such as dimethyl formamide, n-hexane, n-heptane, cyclohexane, cycloheptane; water; or any mixtures of one or more of these solvents; at a temperature ranging from 25−60° C. for about 30 minutes to 2 hours to yield pure Amifampridine. The purification is repeated using different solvents to obtain the desired purity. If required the Amifampridine was purified by using column chromatography to give pure Amifampridine.

Purification of Amifampridine further comprises of providing a solution of Amifampridine using a solvent selected from alcohol (C1-4) or Ketones (C3-6) or organic solvents (C1-8 alkanes, dimethyl formamide) or halogenated organic solvents (Methylene dichloride, Ethylene dichloride) or Ethers (Methyl tertiary butyl ether, tetrahydrofuran, Di-isopropyl ether) or sulphoxides (dimethyl sulphoxide), water or mixtures thereof; acidifying the solution using an acid selected from organic/inorganic acid not limited to formic acid, citric acid, acetic acid, sulfuric acid, phosphoric acid; followed by basification using an organic/inorganic base and isolating the substantially pure Amifampridine having a purity of greater than 99.5%.

Purification of Amifampridine further comprises of providing a solution of Amifampridine using a water. The obtained reaction was heated to 60-85° C., followed by addition of activated carbon and continued stirring for 10 to 30 minutes at 60-85° C. Filtered the material and washed the contents with water, and dried the material at 50-70° C. for 10 hours to 12 hours to yield substantially pure Amifampridine having a purity of greater than 99.5%. This purification helps in controlling the Impurity-C levels to less than 0.25%. If the impurity-C content is greater than the desired level, repeat the same purification to yield desired purity.

The obtained Amifampridine (III) is converted to Amifampridine phosphate comprising preparing a non-alcoholic solution of amifampridine by dissolving amifampridine in a solvent selected from Dimethyl sulfoxide, dimethyl formamide (DMF) or mixtures thereof; followed by addition of phosphoric acid (85% aqueous phosphoric acid); at a temperature ranging from 10 C ° to 40° C.; white solid formation is observed. Optionally adding anti solvent selected from acetone, acetonitrile, methyl ethyl ketone, MIBK, THF, 1,4-Dioxane or mixtures thereof; at a temperature ranging from 15 to 40° C., followed by stirring for a period of 10 minutes to 40 minutes to yield highly pure Amifampridine phosphate having a purity of greater than 99.5% (by HPLC). Filtered the wet cake and washed with a solvent as described above to provide Amifampridine phosphate (I).

In view of this, the present inventors developed an improved process for the preparation of Amifampridine phosphate, by using industrial friendly solvents and reagents, which leads in the formation of good yield with high purity.

In another embodiment, the present invention relates to substantially pure Amifampridine phosphate, wherein in the salt is obtained by the process comprising:
a) preparing a non-alcoholic solution of amifampridine;
b) adding phosphoric acid solution to non-alcoholic solution obtained in step (i); and
c) optionally adding anti solvent to yield highly pure Amifampridine phosphate having a purity of greater than 99.5% (by HPLC).

The substantially pure Amifampridine phosphate obtained as per the present invention is highly pure and having a purity of greater than 99.5%. This purity is achieved by involving a different solvent other than prior reported solvents like water and alcohol. As the solubility of amifampridine is very high in these solvents, it is difficult to isolate the precipitated material, which in parallel leads in low yield process. The present inventors now developed a process for the preparation of substantially pure amifampridine phosphate, using solvent system devoid of water and alcohol, which yields in the formation of highly pure amifampridine phosphate having a triamine impurity of Formula-C less than 0.1%.

The present inventors repeated the process disclosed in the prior process, where in an additional purification of amifampridine is required to remove impurity-C and to meet the ICH guidelines.

In view of this, the present inventors developed an improved process for the preparation of Amifampridine phosphate, by using industrial friendly solvents and reagents, which leads in the formation of good yield with high purity.

Particular aspects of the present specification relate to substantially pure phosphoric acid addition salt of Amifampridine (I) and processes for its preparation thereof. Further, the invention of this application also relates to pharmaceutical compositions comprising of phosphoric acid addition salt of Amifampridine (I), which may be useful in the treatment of various cancerous disorders.

In a further embodiment of the present invention provides phosphoric acid addition salt of Amifampridine represented by Formula (I) may be obtained as a crystalline solid or as an amorphous material.

Phosphoric acid addition salt of Amifampridine can be converted into its amorphous form or its solid dispersion with suitable pharmaceutically acceptable excipients which are preferably selected from polyvinylpyrrolidones (povidones for e.g. Plasdone-K), copolymers of N-vinylpyrrolidone, gums, cellulose derivatives (for e.g. hydroxypropylmethyl celluloses-HPMC), mannitol, cyclodextrins, gelatins, sugars, polyhydroxy alcohols, polyethylene glycols, polyethylene oxides, polyoxyethylene derivatives, polyvinylalcohols, propylene glycol derivatives, or a mixture thereof. Amorphous form of Amifampridine phosphoric acid may be prepared by techniques known to the person skilled in art, such techniques comprising but not limited to dissolving in an organic solvent and suitably utilizing spray drying or film drying methods. In one of the embodiments amorphous form of Amifampridine phosphoric acid was prepared by using rotavapor technique in presence or absence of reduced pressure conditions.

The substantially pure Amifampridine phosphoric acid as described by the present application has been found to be quite stable and easy to handle and store for longer time without any measurable changes in its morphology and physicochemical characteristics, while retaining its properties within the defined limits. This may offer advantages for large scale manufacturing in terms of handling, storage, shelf life and favorable impurity profile. Besides the physical/chemical properties, the novel salt form of the current application further provides advantage in terms of solubility of the drug and hence provides possibility of better bioavailability and pharmacological profile.

The present inventors developed a process for the preparation of Amifampridine phosphate, wherein the reaction course is extremely smooth and achievable at ordinary feasible temperature conditions of 5-85° C., which is not only industrially feasible but also cost effective and provide pure materials/intermediates.

To overcome these serious industrially non-viable process concerns as cited above, the present inventors aimed for a process, which is not only industrially upscale process but also cost effective and least time consuming. The inventors in the present invention found that the use of base in the condensation step makes the reaction to move smoothly at ordinary lower temperatures i.e. at about 30-70° C., which was found to help in avoiding the formation of large number impurities due to unwanted parallel reactions and resulting in recovering purer material.

Drying may be also be performed by any conventional process not limited to spray drying or distillation to remove the solvent. Drying may be performed under reduced pressure conditions also. Reduced pressure conditions may be suitably utilized by person skilled in the art in order to obtain the dried material. The drying may be performed at a temperature ranging from 40-60° C. for time ranging from 6 to 12 hrs depending upon the physical attributes of the end product obtained i.e. Pure Amifampridine phosphate.

The process related impurities that appear in the impurity profile of the Amifampridine phosphate may be substantially removed by the process of the present invention resulting in the formation of highly pure material. The process of the present invention is as summarized in the Scheme-I as represented below:

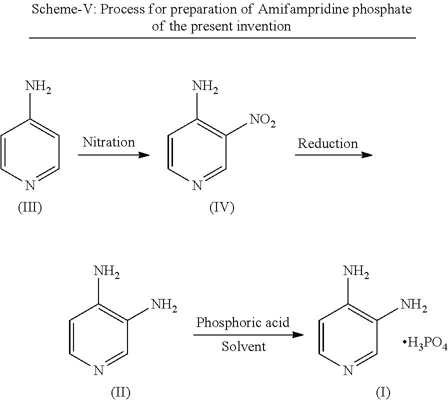

In another embodiment the present invention provides substantially pure Amifampridine phosphate having a purity of greater than 99.5% and total impurities A to D collectively less than 0.5% by HPLC.

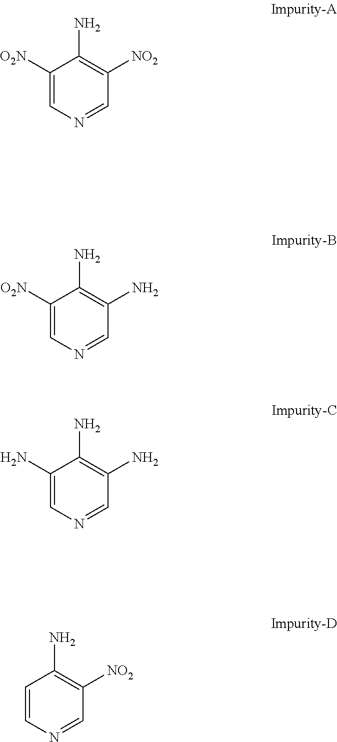 [SMILES: Nc1c([N+](=O)[O-])cncc1[N+](=O)[O-] ; Nc1cncc([N+](=O)[O-])c1N ; Nc1cncc(N)c1N ; Nc1ccncc1[N+](=O)[O-]]

In further embodiment of the present invention provides substantially pure Amifampridine phosphate having a purity of greater than 99.6% by HPLC.

In further embodiment of the present invention provides substantially pure Amifampridine phosphate having a purity of greater than 99.7% by HPLC.

In further embodiment of the present invention provides substantially pure Amifampridine phosphate having a purity of greater than 99.8% by HPLC.

In further embodiment of the present invention provides substantially pure Amifampridine phosphate having a purity of greater than 99.9% by HPLC.

In further embodiment of the present invention provides substantially pure Amifampridine phosphate having a purity of greater than 99.95% by HPLC.

In another embodiment, the Amifampridine phosphate obtained by the processes of the present application may be formulated as solid compositions for oral administration in the form of capsules, tablets, pills, powders or granules. In these compositions, the active product is mixed with one or more pharmaceutically acceptable excipients. The drug substance can be formulated as liquid compositions for oral administration including solutions, suspensions, syrups, elixirs and emulsions, containing solvents or vehicles such as water, sorbitol, glycerine, propylene glycol or liquid paraffin.

The compositions for parenteral administration can be suspensions, emulsions or aqueous or non-aqueous sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g. using a bacteriological filter, by incorporating sterilizing agents in the composition, by irradiation or by heating. They may be prepared in the form of sterile compositions, which can be dissolved at the time of use in sterile water or any other sterile injectable medium.

Pharmaceutically acceptable excipients used in the compositions comprising Amifampridine phosphate obtained as per the present application process—include, but are but not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, pre-gelatinized starch and the like; disintegrants such as starch, sodium starch glycolate, pregelatinized starch, Croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants, waxes and the like. Other pharmaceutically acceptable excipients that are of use include but not limited to film formers, plasticizers, colorants, flavoring agents, sweeteners, viscosity enhancers, preservatives, antioxidants and the like.

Pharmaceutically acceptable excipients used in the compositions derived from Amifampridine phosphate of the present application may also comprise to include the pharmaceutically acceptable carrier used for the preparation of solid dispersion, wherever utilized in the desired dosage form preparation.

The following examples illustrate the nature of the invention and are provided for illustrative purposes only and should not be construed to limit the scope of the invention.

### EXAMPLES

### Example 1

### Preparation of 3-Nitro 4-Amino Pyridine (III)

4-amino pyridine (50 gm, 0.5319 mol) was added in to reaction flask containing Sulfuric acid (350 mL) slowly at a temperature of 5 to 20° C. To the reaction mixture Potassium nitrate (57.5 gram)) was added for a period of 30 minutes at 15-20° C. under stirring. Raised temperature to room temperature then heated up to 40-45° C. under stirring. Maintain the temperature for 6 hours under stirring. Cooled the reaction mixture to room temperature and then quenched to another reaction containing water (1750 ml) was added. Cool to 0-5° C. and adjust pH of the reaction mass to 7.0-8.0 using aqueous ammonia solution. Filtered the material and washed with water (20 ml). Dried the material under vacuum at 50° C. for 14 to 16 hours till constant weight appears to yield the tilted compound. The obtained compound is slurried using methanol (250 ml) for a period of 2 hours to 3 hours at 25-30° C., suck dried the material and dried the material at under vacuum at 50° C. for a period of 14 to 16 hours to yield highly pure title compound

Yield: 47.6 gm

Chromatographic purity (By HPLC): 96.2%

### Example 2

### Preparation of 3, 4-Diamino Pyridine (Amifampridine)

Methanol (70 ml) and Water (21.0 ml) was charged in to reaction flask containing 4-amino-3-nitro pyridine (46.0 g, 0.33 mol.) The reaction mixture was heated to 50-55° C. under stirring. Sodium sulfide solution (Na₂S 104.5 gm in 170 ml D.M. Water) was slowly added in to reaction mass at 50-55° C. for 30 min. raise the temperature for 60-65° C. and continued stirring for 3 hours to 4 hours. Cooled the reaction mass to 0-5° C. and then stirred the reaction mass for 1-2 hours. Filtered the material and washed with water (46 ml). Suck dry the material and air dried the material at 25-30° C. for a period of 10 to 12 hours to yield to yield the tilted compound.

Yield: 35.3 gm (Wet)

Chromatographic Purity (By HPLC): 97.7%

### Example 3

### Purification of Amifampridine (II)

Water (318 ml) was charged in to reaction flask containing Crude Amifampridine (35.3 gm). The reaction mass was heated to 80-85° C. to get clear solution. Activated carbon (10%) was added and stirred the contents for 10 to 15 minutes at 80-85° C. Filtered through celite bed and washed with water (20 ml). Cooled the obtained reaction mass to 0-5° C. and continued stirring for 1 hour to 2 hours. Filtered the material and washed with chilled water. Dried the material at 50-55° C. for 10 hours to 12 hours to yield pure Amifampridine. If desired repeat the purification to achieve impurity-C less than 0.25%.

Yield: 13.5 gm (Wet)

Chromatographic Purity (By HPLC): 99.80%

### Example 4

### Preparation of Amifampridine Phosphate (I)

Amifampridine (10 gm) was charged in to reaction flask containing DMSO (40.0 ml). cool the reaction mass to 15-20° C. ortho Phosphoric acid (17 ml) was added under stirring at 15-20° C. Charge acetone (167.0 ml) into the reaction mass at 20-25° C. and continue stirring for 10-15 minutes. Cooled the reaction mass to 0-5° C. and continue stirring for 3 to 4 hours at 0-5° C. Filtered the material and washed with Acetone (100.0 ml). Dried the material under vacuum at 40-45° C. for 6 hours to 10 hours to yield highly pure Amifampridine phosphate.

Yield: 16.6 gm

Chromatographic Purity (By HPLC): 99.90%

Phosphate content: 45.95%

Water Content (By KF): 0.54%

Total impurities (By HPLC): 0.10%

### Example 5

### Preparation of Amifampridine Phosphate (I)

Amifampridine (1.5 gm) was charged in to reaction flask containing DMSO (6.0 ml). cool the reaction mass to 15-20° C. ortho Phosphoric acid (2.5 ml) was added under stirring at 15-20° C. Cooled the reaction mass to 0-5° C. and continue stirring for 3 to 4 hours at 0-5° C. Filtered the material. Dried the material under vacuum at 40-45° C. for 6 hours to 10 hours to yield highly pure Amifampridine phosphate.

Yield: 1.65 gm

Chromatographic Purity (By HPLC): 99.94%

Phosphate content: 45.88%

Total impurities (By HPLC): 0.056%

While the foregoing pages provide a detailed description of the preferred embodiments of the invention, it is to be understood that the description and examples are illustrative only of the principles of the invention and not limiting. Furthermore, as many changes can be made to the invention without departing from the scope of the invention, it is intended that all material.

We claim:

1) An improved process for the preparation of substantially pure Amifampridine phosphate of Formula (I) 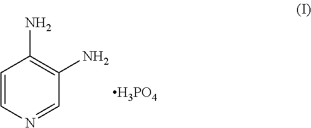
comprising the steps of:
a) reacting 4-amino pyridine (III) with a nitrating agent in presence/absence of a solvent to yield 4-Amino-3-Nitro Pyridine (IV); 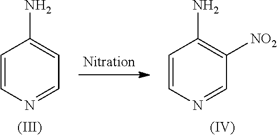
b) reacting 4-Amino-3-Nitro Pyridine (IV) with a reducing agent to provide Amifampridine (II); and 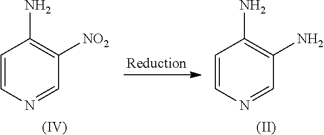
c) converting Amifampridine to Amifampridine phosphate salt comprises:
i. preparing a non-alcoholic solution of amifampridine;
ii. adding phosphoric acid solution to non-alcoholic solution obtained in step (i); and
iii. optionally adding anti solvent to yield highly pure Amifampridine phosphate having a purity of greater than 99.5% (by HPLC).

2) An improved process for the preparation of substantially pure Amifampridine phosphate according to claim 1, wherein nitration is carried out in presence of nitrating agent selected from nitric acid, nitric acid+sulfuric acid, sodium nitrate, potassium nitrate, silver nitrate, Methyl nitrate, nitric acid+sulfuric acid+urea.

3) An improved process for the preparation of substantially pure Amifampridine phosphate according to claim 1, wherein solvent used in step a) or step b) is selected from acids such as sulfuric acid, acetic acid; nitriles such acetonitrile; nitro methane; alcohols such as C1-5 alcohols; Glycol ethers such as 2-Butoxyethanol, Diglyme, Dimethoxyethane, 2-Ethoxy ethanol, 2-(2-Ethoxyethoxy)ethanol, 2-Methoxyethanol, 2-(2-Methoxyethoxy) ethanol, Octaethylene glycol monododecyl ether, Pentaethylene glycol monododecyl ether, Phenoxyethanol, Propylene glycol methyl ether acetate, Tetraethylene glycol dimethyl ether, Triethylene glycol, Triethylene glycol dimethyl ether; water or mixtures thereof.

4) An improved process for the preparation of substantially pure Amifampridine phosphate according to claim 1, wherein reducing agent is selected from hydrogen sulfide, sodium sulfide, potassium sulfide, sodium hydrogen sulfide, Zn/Magnesium in presence of Hydrazine hydrate or a mixture thereof.

5) An improved process for the preparation of substantially pure Amifampridine phosphate according to claim 1, wherein non-alcoholic solution of amifampridine is prepared by dissolving amifampridine in a solvent selected from dimethyl sulfoxide, dimethyl formamide.

6) An improved process for the preparation of substantially pure Amifampridine phosphate according to claim 1, wherein anti solvent selected from acetone, acetonitrile, methyl ethyl ketone, MIBK, THF, 1,4-Dioxane or mixtures thereof.

7) Substantially pure Amifampridine phosphate, wherein in the salt is obtained by the process comprising:
a) preparing a non-alcoholic solution of amifampridine;
b) adding phosphoric acid solution to non-alcoholic solution obtained in step (i); and
c) optionally adding anti solvent to yield highly pure Amifampridine phosphate having a purity of greater than 99.5% (by HPLC).

8) Substantially pure Amifampridine phosphate according to claim 7, wherein non-alcoholic solution of amifampridine is prepared by dissolving amifampridine in a solvent selected from dimethyl sulfoxide, dimethyl formamide.

9) Substantially pure Amifampridine phosphate according to claim 7, wherein anti solvent selected from acetone, acetonitrile, methyl ethyl ketone, MIBK, THF, 1,4-Dioxane.

10) Substantially pure Amifampridine phosphate, having a purity of greater than 99.5% and total impurities A to D collectively less than 0.5% by HPLC and specifically impurity-C less than 0.1%. 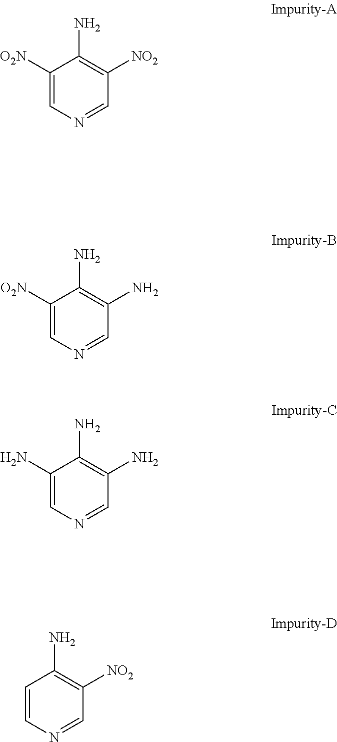 [SMILES: Nc1c([N+](=O)[O-])cncc1[N+](=O)[O-] ; Nc1cncc([N+](=O)[O-])c1N ; Nc1cncc(N)c1N ; Nc1ccncc1[N+](=O)[O-]]
